(19) United States
(12) Patent Application Publication
(10) Pub. No.: US 2021/0038591 A1
(43) Pub. Date: Feb. 11, 2021

(54) VIRAL VECTOR TRANSDUCTION

(71) Applicant: Oxford University Innovation Limited, Oxford (GB)

(72) Inventor: Kanmin XUE, Oxford (Headington) (GB)

(21) Appl. No.: 16/967,776

(22) PCT Filed: Feb. 8, 2019
(86) PCT No.: PCT/EP2019/053193

§ 371 Date: Aug. 6, 2020

(30) Foreign Application Priority Data
Feb. 9, 2018 (GB) 1802122.0

(51) Int. Cl. A61K 31/4706; A61P 33/06; C12N 15/86

(52) U.S. Cl. CPC A61K 31/4706; A61P 33/06; C12N 2750/14133; C12N 2750/14143; C12N 15/86

(57) ABSTRACT

The invention relates to a method of improving the efficiency of transduction of viral vectors into cells, wherein the method comprises administering to a cell an antimalarial agent and a viral vector. The invention also relates to a composition comprising an antimalarial agent and a viral vector for use in increasing the efficiency of transduction of the viral vectors. The invention further relates to use of the method and compositions of the invention to treat a disease or disorder.

The present invention relates to a method or product for improving the efficacy of viral vector transduction into a cell, and in particular to the use of the method or product of the invention to improve the efficacy of gene therapy.

Gene therapy is the therapeutic delivery of nucleic acid into a patient's cells in order to treat or cure a disease. Over the past 3 decades gene therapy has advanced dramatically, with the in vivo delivery of therapeutic adeno-associated viral (AAV) vectors to the retina, liver and nervous system resulting in a clinical improvement in patients with congenital blindness, haemophilia B and spinal muscular atrophy.

Whilst success is being seen with gene therapy techniques there are still a number of hurdles preventing increased use of this therapeutic method. One is the efficiency of viral vector uptake by target cells. The efficacy of gene therapy is, in general, dependent upon adequate and efficient delivery of donated DNA in order to correct a genetic defect, this process is usually mediated by viral vectors. A sufficient proportion of target cells needs to be transduced by the viral vector in order to slow down or stop the progression of disease, and this needs to be achieved with a sufficiently low vector dose to avoid the risk of immunological or inflammatory responses to the viral vector.

The following example demonstrates the need to improve viral vector transduction in order to improve the efficacy of gene therapy. Clinical trials using gene therapy to treat inherited retinal dystrophies (such as Leber congenital amaurosis and choroideremia) using adeno-associated viral (AAV) vectors demonstrated safety and promising early efficacy. However, the magnitude of therapeutic effect is expected to be dependent on the proportion of the non-dividing retinal cells successfully transduced by AAV vectors. High doses of vector (in the order of 10¹⁰-10¹¹ genome particles) are required to transduce retinal pigment epithelial (RPE) and photoreceptor cells even within the limited confines of a subretinal injection, but this is very close to the dose that could induce an inflammatory response. AAV vectors also show cell type-specific tropism whereby bipolar, Müller and ganglion cells within the retina are poorly transduced, which prevents application of gene therapy to diseases affecting those cell types. Moreover, AAV vectors that appear to transduce mouse retina with high efficiency in pre-clinical studies do not necessarily achieve equivalent efficiency in primate (including human) retina. AAV vector gene therapy for inherited retinal dystrophies should ideally achieve a transduction rate of >50% of the target retinal cells, since female carriers of X-linked retinal dystrophies (e.g. choroideremia and retinitis pigmentosa associated with RPGR mutations) are generally unaffected despite 50% random X-chromosome inactivation. A transduction rate of <50% may be associated with continued disease progression despite gene therapy due to degeneration of the untransduced cells (with secondary degeneration of the neighbouring cells). Therefore, to improve the efficacy of gene therapy approaches which use viral vectors there is a need to improve the rate of transduction into cells by viral vectors, and in particular AAV vectors.

The present invention provides a method of improving the efficiency of transduction of viral vectors into cells, wherein the method comprises administering to a cell an antimalarial agent and a viral vector. The antimalarial agent and viral vector may be administered simultaneously, sequentially or separately.

The Antimalarial Agent

The antimalarial agent may be selected from the group comprising 4-aminoquinolines (such as hydroxychloroquine, chloroquine and amodiaquine), 8-aminoquinolines (such as primaquine, pamaquine and tafenoquine), mefloquine, quinine, mepacrine, atovaquone, doxycycline, or a salt or derivative thereof.

Preferably the antimalarial agent is a quinoline compound, such as hydroxychloroquine, chloroquine, mefloquine, amodiaquine, quinine, pamaquine, primaquine, mepacrine, or a salt, or a derivative thereof. Preferably the antimalarial agent is hydroxychloroquine, chloroquine, mefloquine, or salt or derivative thereof, or a combination thereof. Preferably the antimalarial agent is hydroxychloroquine.

Hydroxychloroquine or HCQ (chemical name: 2-[{4-[(7-chloroquinolin-4-yl) amino]pentyl}(ethyl)amino]ethanol) has been known since the early 1950s (U.S. Pat. No. 2,546,658). It was initially developed as an antimalarial drug and sold as the sulfate salt by Sanofi Aventis under the trade name Plaquenil®. Oral hydroxychloroquine sulfate is also used for the treatment of rheumatoid arthritis and inflammatory skin diseases, including systemic lupus erythematosus.

The chemical structure of hydroxychloroquine is as shown below.

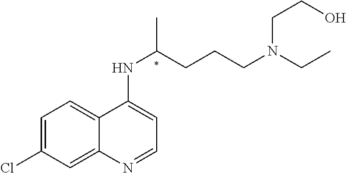 [SMILES: CCN(CCO)CCCC(C)Nc1ccnc2cc(Cl)ccc12]

The compound has a chiral centre at the carbon atom that is identified with an asterisk and hence can exist in two enantiomeric forms, (R)-(−)-2-[{4-[(7-chloroquinolin-4-yl)amino]pentyl}(ethyl)amino]ethanol) [hereinafter (R)-(−)-hydroxychloroquine] and (S)-(+)-2-[{4-[(7-chloroquinolin-4-yl)amino]pentyl}(ethyl)amino]ethanol) [hereinafter (S)-(+)-hydroxychloroquine].

Hydroxychloroquine can therefore exist as a racemate consisting of a 1:1 mixture of two enantiomers, substantially the single (R)-(−)-hydroxychloroquine or substantially the single (S)-(+)-hydroxychloroquine. Commercially available hydroxychloroquine may be a racemic mixture of the two enantiomers.

Methods for the preparation of hydroxychloroquine racemate or isomers are known in the art. For example, hydroxychloroquine can be prepared according to U.S. Pat. No. 2,546,658 and U.S. Pat. No. 5,314,894.

Chloroquine or CQ (chemical name: 2-[{4-[(7-chloroquinolin-4-yl)amino]pentyl}(ethyl)amino]ethanol) is a member of the drug class 4-aminoquinoline and is also used as an antimalarial drug. It may be sold under the trade name Resochine®, Dawaquin® or Chlorquine FNA®.

The chemical structure of chloroquine is as shown below.

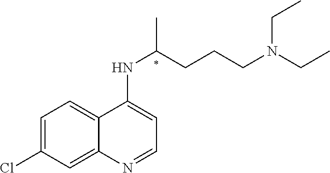 [SMILES: CCN(CC)CCCC(C)Nc1ccnc2cc(Cl)ccc12]

Like hydroxychloroquine, chloroquine has a chiral centre at the carbon atom that is identified with an asterisk and hence can exist in two enantiomeric forms. Chloroquine can therefore exist as a racemate consisting of a 1:1 mixture of two enantiomers, substantially the single (R)-(−)-chloroquine or substantially the single (S)-(+)-chloroquine. Commercially available chloroquine may be a racemic mixture of the two enantiomers.

Mefloquine is a quinolinemethanol derivative, commonly sold under the brand name Lariam®, which is used to treat or prevent malaria. The chemical structure of mefloquine is as shown below.

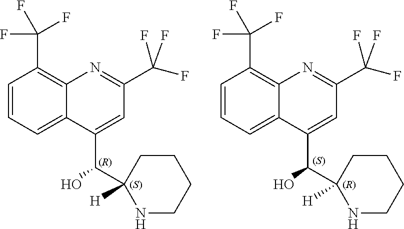

Mefloquine is a chiral molecule with two asymmetric carbon centres, which means it has four different stereoisomers. The drug is currently manufactured and sold as a racemate of the (R,S)- and (S,R)-enantiomers by Hoffman-LaRoche. Essentially, it is two drugs in one. Plasma concentrations of the (−)-enantiomer are significantly higher than those for the (+)-enantiomer, and the pharmacokinetics between the two enantiomers are significantly different. The (+)-enantiomer has a shorter half-life than the (−)-enantiomer.

The Viral Vector

The viral vector may be designed to deliver a cargo to a cell. The cargo may be a therapeutic transgene product (DNA). The intention may be that the therapeutic transgene will remain in the cell nucleus (either as an episome or integrate into the host genomic DNA) and express the desired gene product.

The viral vector may be selected from the group comprising an adeno-associated virus (AAV), an adenovirus, a retrovirus, a lentivirus, a vaccinia/poxvirus, or a herpesvirus (e.g., herpes simplex virus (HSV)). In a preferred embodiment, the vector is an adeno-associated viral (AAV) vector. In an embodiment, the vector is an adeno-associated viral (AAV) vector ad the antimalarial agent is hydroxychloroquine.

Multiple serotypes of adeno-associated virus (AAV), including 12 human serotypes (AAV1, AAV2, AAV3, AAV4, AAV5, AAV6, AAV7, AAV8, AAV9, AAV10, AAV11, and AAV12) and more than 100 serotypes from non-human primates have now been identified. The serotype of the inverted terminal repeats (ITRs) or the capsid sequence of the AAV vector may be selected from any known human or non-human AAV serotype. In some embodiments a pseudotyping approach is employed, wherein the genome of one ITR serotype is packaged into a different serotype capsid.

Different AAV vector serotypes may have different tissue/cell type specificity, and an appropriate serotype may be selected for use with a particular tissue/cell type. In an embodiment AAV2/2 may be used to transduce RPE cells. In another embodiment AAV2/5 and/or AAV2/8 may be used to transduce photoreceptors.

The invention may use a dual AAV vector approach, in which the aim is to deliver a large transgene using two different AAV vectors, for example in the treatment of Stargardt disease (ABCA4) dual AAV vectors are required as the gene is too big for one vector. In such approaches the efficiency of vector transduction into cells is even more important.

The method of the invention may be practised on a mammal, preferably the mammal is selected from the group comprising a human, a non-human primate, a horse, a cat, a dog, a sheep, a goat, a cow, a pig, a mouse or a rat.

The antimalarial agent and/or viral vector may be administered systemically (for example, orally, intravenously, sublingually and transdermally) or locally (for example, intraperitoneally, intrathecally, intraventricularly or by direct injection into a tissue or organ). Preferably, for clinical simplicity, the antimalarial agent and the viral vector are administered in the same way.

The antimalarial agent and/or viral vector may be administered in a single administration or in several administrations. In a preferred embodiment the antimalarial agent and viral vector are each administered only once, preferably within about a week, within about 5 days, within about 3 days, within about 2 days, within about 24 hours, within about 12 hours, within about 6 hours, within about 1 hour, within about 30 minutes, within about 10 minutes of each other. The time between the delivery of the antimalarial agent and the viral vector may be different for different tissue/cell types and for different applications. The delivery protocol may be optimised using routine techniques.

Preferably the antimalarial agent and viral vector are administered simultaneously, separately or sequentially, preferably to the same cells, tissue or organ.

Simultaneous administration, within the meaning of the present invention means that the antimalarial agent and the viral vector are administered by the same route and at the same time or at substantially the same time. The antimalarial agent and the viral vector may be in the same composition.

Separate administration, within the meaning of the present invention means that the antimalarial agent and the viral vector are administered at the same time or at substantially the same time by different routes.

Sequential administration, within the meaning of the present invention means that the antimalarial agent and the viral vector are administered at different times, the administration route being identical or different.

The antimalarial agent and/or viral vector may be administered at the intended site of action, for example into the tissue or organ where transduction of the viral vector into the target cells is required. Preferably both the antimalarial agent and viral vector are administered at the intended site of action. Preferably the antimalarial agent and viral vector are administered by injection, the antimalarial agent and viral vector may be co-administered in a single injection or administered in separate injections.

The method of the invention may be used to improve the transduction of viral vectors into cells of any tissue or organ. The cells may be dividing or non-dividing cells. The cell types may include cells of any tissue, the tissue may be the interstitial or luminal space of tissues in the lungs, the trachea, the skin, the muscles, the brain, the liver, the heart, the spleen, the bone marrow (in vivo or ex vivo), the thymus, the bladder, the lymphatic system, the blood, the pancreas, the stomach, the kidneys, the ovaries, the testicles, the rectum, the peripheral or central nervous system (CNS), the eyes, the lymphoid organs, the cartilage, or the endothelium. The cell type may be a cell type found in the eye, for example cells of the retinal pigment epithelium (RPE) or photoreceptors. Alternatively, the cell type may be a type of cell found in the CNS.

Expression of a cargo introduced by viral vectors in a method of the invention may be controlled by cell/tissue/organ specific regulatory elements, such as tissue-specific transcription promoters. Alternatively, the expression of the transgene may be controlled by a ubiquitous transcription promoter.

The method of the invention may be used to deliver viral vectors carrying a cargo. The cargo may comprise a transgene or a part thereof intended to treat a disease or disorder. Alternatively, or additionally, the cargo comprise at least one of the components needed to facilitate CRISPR gene editing, such as the Cas9 enzyme. Alternatively, or additionally, the cargo comprise the components needed to produce an inhibitory RNA or a Mirtron. In another embodiment the cargo may comprise one or more components needed to deliver an optogenetic therapy (e.g. a photosensitive opsin) or system to a cell, tissue or organ.

In an alternative embodiment, the method of the invention may be used to increase the level of gene expression from an existing AAV construct with a low-efficiency promoter (e.g. an endogenous promoter as opposed to a ubiquitous promoter).

In another aspect the method of the invention may provide a method to increase the yield of recombinant AAV particles during a production run of the viral vector. This may be achieved by adding an antimalarial agent to a producer cell line, e.g. HEK-293 cells, within protocols known in the art.

The method of the invention may be used to treat any disorder or disease of a mammal. The method of the invention may use gene therapy to treat a disorder or disease of a mammal. The method of the invention may use gene editing and block-and-replace therapies to treat a disorder or a disease of a mammal.

The method of the invention may be used to treat an ocular disorder or disease, or to enhance the gene therapy treatment of an ocular disorder or disease. The ocular disorder or disease may be selected from group comprising severe early-onset retinal degenerations (Leber's congenital amaurosis), congenital colour or night vision defects (e.g. achromatopsia), retinitis pigmentosa (autosomal dominant, autosomal recessive, X-linked, or mitochondrial inheritance), macular dystrophies (e.g. Stargardt's disease, Best's disease), choroideremia, Doyne's retinal dystrophy, cone dystrophies, X-linked retinoschisis, retinal ciliopathies (e.g. Usher's syndrome), wet (or neovascular) age-related macular degeneration, dry age-related macular degeneration, diabetic retinopathy (e.g. diabetic maculopathy, proliferative diabetic retinopathy), cystoid macular oedema, central serous retinopathy, inherited retinal detachment (e.g. connective tissue disorders, such as Stickler syndrome), uveitis (e.g. CAPN5-associated hereditary uveitis), inherited optic neuropathies (e.g. Leber's optic neuropathy), glaucoma, and inherited corneal dystrophies. The method of the invention may also enhance the efficacy of other gene therapy rescue strategies for retinal degenerations, such as dual-vector gene therapy (e.g. using multiple AAV vectors to deliver fragments of a large transgene), optogenic therapies (e.g. using AAV to deliver melanopsin or other opsin genes), neurotrophic factor therapies (e.g. using AAV to deliver ciliary neutrophic factor, CNTF, or other survival/anti-apoptotic factors to the retina or optic nerve), gene editing (e.g. using AAV to deliver CRISPR-cas9 constructs), and ‘block-and-replace’ therapies (e.g. using AAV to deliver inhibitory RNAs or Mitron-elements together with a transgene). The antimalarial agent may be administered directly to the eye, for example by subretinal, suprachoroidal, intravitreal, peri-ocular or anterior chamber injection, as an adjunct to AAV retinal gene therapy. The AAV vector may also be administered directly to the eye, for example by subretinal, suprachoroidal, intravitreal, peri-ocular or anterior chamber injection.

In an alternative embodiment the method of the invention may be used to treat a CNS condition, and the AAV vector and/or the antimalarial agent may be administered by direct spinal cord injection and/or intracerebral administration. In some embodiments, the intracerebral administration is at a site selected from the group consisting of the cerebrum, medulla, pons, cerebellum, intracranial cavity, meninges surrounding the brain, dura mater, arachnoid mater, pia mater, cerebrospinal fluid (CSF) of the subarachnoid space surrounding the brain, deep cerebellar nuclei of the cerebellum, ventricular system of the cerebrum, subarachnoid space, striatum, cortex, septum, thalamus, hypothalamus, and the parenchyma of the brain. In some embodiments, the administration is by intracerebroventricular injection into at least one cerebral lateral ventricle. In some embodiments, the administration is by intrathecal injection in the cervical, thoracic, and/or lumbar region. In some embodiments, the administration is by intrastriatal injection. In some embodiments, the administration is by intrathalamic injection. In some embodiments, the administration is by intraparenchymal injection. In some embodiments, the administration comprises direct spinal cord injection, intracranial, and/or intracerebral administration.

The method of the invention may be used to treat a disorder or disease of the CNS. The disorder or disease may be a lysosomal storage disease (LSD), Huntington's disease, epilepsy, Parkinson's disease, Alzheimer's disease, stroke, corticobasal degeneration (CBD), corticobasal ganglionic degeneration (CBGD), frontotemporal dementia (FTD), multiple system atrophy (MSA), progressive supranuclear palsy (PSP) or cancer of the brain. In some embodiments, the disorder is a lysosomal storage disease selected from the group consisting of aspartylglusoaminuria, Fabry, Infantile Batten Disease (CNL1), Classic Late Infantile Batten Disease (CNL2), Juvenile Batten Disease (CNL3), Batten form CNL4, Batten form CNL5, Batten form CNL6, Batten form CNL7, Batten form CNL8, cystinosis, Farber, fucosidosis, Galactosidosialidosis, Gaucher disease type 1, Gaucher disease type 2, Gaucher disease type 3, GM1 gangliosidosis, Hunter disease, Krabbe disease, α-mannosidosis disease, β-mannosidosis disease, Maroteaux-Lamy, metachromatic leukodystrophy disease, Morquio A, Morquio B, mucolipidosisII/III disease, Niemann-Pick A disease, Niemann-Pick B disease, Niemann-Pick C disease, Pompe disease, Sandhoff disease, SanfiUipo A disease, SanfiUipo B disease, SanfiUipo C disease, SanfiUipo D disease, Schindler disease, Schindler-Kanzaki, sialidosis, Sly disease, Tay-Sachs disease, and Wolman disease. In some embodiments, the disorder of the CNS is Huntington's disease, Parkinson's disease, Alzheimer's disease, or frontotemporal dementia.

The method of the invention (adjunctive use of an anti-malarial agent such as HCQ or CQ) may allow the proportion of cells transduced by the viral vector in a target cell population to be increased by 1, 2, 3 fold, or more, compared with using the viral vector alone. To slow down or halt disease progression in a monogenic recessive disease, preferably at least 50%, more preferably at least 60%, of cells which are in contact with the AAV vector and the antimalarial agent, are transduced by the viral vector.

The method of the invention may result in an increase in the efficiency of transduction of the viral vector. In an embodiment, the increase in the efficiency of transduction may have a clinical benefit to a subject. The rate of transduction may be increased by less than 1 fold, preferably at least about 0.5 fold, at least about 1 fold, preferably an at least about 1.5 fold preferably at least about 2 fold, preferably at least about 3 fold, preferably at least about 4 fold, preferably at least about 5 fold, preferably at least about 6 fold, preferably at least about 7 fold, preferably at least about 8 fold, preferably at least about 9 fold, preferably at least about 10 fold or more increase in the rate of viral vector transduction. Preferably the fold increase in the rate of viral vector transduction is between about 1 and about 5 fold. The fold increase in viral vector transduction observed as a result of the method of the invention may be determined in vitro.

The increase in the efficiency of transduction of a viral vector may alternatively be measured in terms of the percentage increase, the increase may be at least about 1%, at least about 2%, at least about 3%, at least about 4%, at least about 5% or more. The increase in the efficiency of transduction may be about 10% or more, about 20% or more, about 30% or more, about 40% or more, about 50% or more, about 60% or more, about 70% or more, about 80% or more, about 90% or more, about 100% or more, about 200% or more, about 300% or more, about 400% or more, or about 500% or more.

The percentage or fold increase in transduction may be determined by comparison to the rate of transduction observed in the absence of the antimalarial agent.

In a method of the invention the antimalarial agent, in particular HCQ, is used at a concentration of between about 1 μM and about 30 μM, between about 3 μM and about 30 μM, between about 3 μM and about 25 μM, between about 5 μM and about 25 μM, between about 10 μM and about 30 μM, between about 10 μM and about 25 μM, between about 5 μM and about 20 μM, between about 10 μM and about 20 μM, and between about 15 μM and about 20 μM. The antimalarial agent may be used at a concentration of less than about 30 μM, less than about 25 μM, or less than about 20 μM. The concentration of antimalarial agent used is a balance between increasing viral vector transduction and toxicity to the cells.

In a preferred embodiment the viral vector is an AAV vector, and preferably an at least 2 fold increase in viral vector transduction is observed in the presence of an antimalarial agent, wherein the antimalarial agent is preferably HCQ or CQ. The HCQ or CQ may be used at a concentration of about between about 1 μM and about 30 μM, between about 3 μM and about 30 μM, between about 3 μM and about 25 μM, between about 5 μM and about 25 μM, between about 10 μM and about 30 μM, between about 10 μM and about 25 μM, between about 5 μM and about 20 μM, between about 10 μM and about 20 μM, and between about 15 μM and about 20 μM. The antimalarial agent may be used at a concentration of less than about 30 μM, less than about 25 μM, or less than about 20 μM. In this preferred embodiment the vector and antimalarial agent may be administered directly to the eye of a subject.

In a further aspect, the invention provides the use of the method of the invention to treat a disease or disorder in a subject. The disease or disorder may be a disease or disorder of the eye.

In another aspect the invention provides a composition comprising a viral vector and an antimalarial agent. The composition may be a pharmaceutical composition, which may further comprise a pharmaceutically acceptable carrier, diluent or excipient. In an embodiment the pharmaceutical composition is for intraocular administration.

In an embodiment the invention provides a pharmaceutical composition comprising an AAV vector, one or more of hydroxychloroquine, chloroquine, and mefloquine and a pharmaceutically acceptable carrier, diluent or excipient. The pharmaceutical composition may comprise an AAV vector and hydroxychloroquine and a pharmaceutically acceptable carrier, diluent or excipient. The pharmaceutical composition may be for intraocular administration.

In a further aspect, the invention provides a composition or pharmaceutical composition according to the invention for use in the treatment of a disease or disorder. The disease or disorder may be a disease or disorder of the eye.

The ocular disorder or disease may be selected from group comprising severe early-onset retinal degenerations (Leber's congenital amaurosis), congenital colour or night vision defects (e.g. achromatopsia), retinitis pigmentosa (autosomal dominant, autosomal recessive, X-linked, or mitochondrial inheritance), macular dystrophies (e.g. Stargardt's disease, Best's disease), choroideremia, Doyne's retinal dystrophy, cone dystrophies, X-linked retinoschisis, retinal ciliopathies (e.g. Usher's syndrome), wet (or neovascular) age-related macular degeneration, dry age-related macular degeneration, diabetic retinopathy (e.g. diabetic maculopathy, proliferative diabetic retinopathy), cystoid macular oedema, central serous retinopathy, inherited retinal detachment (e.g. connective tissue disorders, such as Stickler syndrome), uveitis (e.g. CAPN5-associated hereditary uveitis), inherited optic neuropathies (e.g. Leber's optic neuropathy), glaucoma, and inherited corneal dystrophies. The method of the invention may also enhance the efficacy of other gene therapy rescue strategies for retinal degenerations, such as dual-vector gene therapy (e.g. using AAV to deliver multiple fragments of a large transgene), optogenic therapies (e.g. using AAV to deliver melanopsin or other opsin genes), neurotrophic factor therapies (e.g. using AAV to deliver ciliary neutrophic factor, CNTF, or other survival/anti-apoptotic factors to the retina or optic nerve), gene editing (e.g. using AAV to deliver CRISPR-cas9 constructs), and ‘block-and-replace’ therapies (e.g. using AAV to deliver inhibitory RNAs or Mitron-elements together with a transgene).

In another aspect, the invention may provide a viral vector, such as an AAV vector, and an antimalarial agent, such as hydroxychloroquine, for use in the treatment of a disease or disorder in a subject. The disease or disorder may be a disease or disorder of the eye. The antimalarial agent may be any agent described herein. The viral vector may be any vector described herein. The viral vector and antimalarial agent may be provided in the same or different compositions. If in different compositions the viral vector and antimalarial agent may be intended to by administered simultaneously, sequentially or separately. In an embodiment the invention provide an AAV vector and hydroxychloroquine for use in the treatment of a disease or disorder in a subject.

The AAV vector and/or the antimalarial agent, and/or the pharmaceutical composition, and/or the method of the invention, may be administered to the eye of a subject by subretinal, direct retinal, suprachoroidal or intravitreal injection

In an embodiment the AAV vector may be administered by intraocular injection whilst the antimalarial agent is administered separately by systemic administration. The antimalarial agent may be hydroxychloroquine.

In an alternative embodiment the AAV vector and/or the antimalarial agent and/or the pharmaceutical composition may be administered by direct spinal cord injection and/or by intracerebral administration. Alternatively, the AAV vector may be administered by direct spinal cord injection and/or intracerebral administration whilst the antimalarial agent is administered separately by systemic administration.

The invention may further provide a method of treating a disease or disorder of the eye comprising administering an AAV vector and an antimalarial agent, or a pharmaceutical composition according to the invention, to a subject in need thereof. The disorder or disease of the eye may be selected from group comprising severe early-onset retinal degenerations (Leber's congenital amaurosis), congenital colour or night vision defects (e.g. achromatopsia), retinitis pigmentosa (autosomal dominant, autosomal recessive, X-linked, or mitochondrial inheritance), macular dystrophies (e.g. Stargardt's disease, Best's disease), choroideremia, Doyne's retinal dystrophy, cone dystrophies, X-linked retinoschisis, retinal ciliopathies (e.g. Usher's syndrome), wet (or neovascular) age-related macular degeneration, dry age-related macular degeneration, diabetic retinopathy (e.g. diabetic maculopathy, proliferative diabetic retinopathy), cystoid macular oedema, central serous retinopathy, inherited retinal detachment (e.g. connective tissue disorders, such as Stickler syndrome), uveitis (e.g. CAPN5-associated hereditary uveitis), inherited optic neuropathies (e.g. Leber's optic neuropathy), glaucoma, and inherited corneal dystrophies. The method of the invention may also enhance the efficacy of other gene therapy rescue strategies for retinal degenerations, such as dual-vector gene therapy (e.g. using AAV to deliver multiple fragments of a large transgene), optogenic therapies (e.g. using AAV to deliver melanopsin or other opsin genes), neurotrophic factor therapies (e.g. using AAV to deliver ciliary neutrophic factor, CNTF, or other survival/anti-apoptotic factors to the retina or optic nerve), gene editing (e.g. using AAV to deliver CRISPR-cas9 constructs), and ‘block-and-replace’ therapies (e.g. using AAV to deliver inhibitory RNAs or Mitron-elements together with a transgene). The invention may further provide a method of treating a disease or disorder of the CNS comprising administering an AAV vector and an antimalarial agent, or a pharmaceutical composition according to the invention, to a subject in need thereof. The disease or disorder of the CNS may be a lysosomal storage disease (LSD), Huntington's disease, epilepsy, Parkinson's disease, Alzheimer's disease, stroke, corticobasal degeneration (CBD), corticogasal ganglionic degeneration (CBGD), frontotemporal dementia (FTD), multiple system atrophy (MSA), progressive supranuclear palsy (PSP) or cancer of the brain. In some embodiments, the disorder is a lysosomal storage disease selected from the group consisting of aspartylglusoaminuria, Fabry, Infantile Batten Disease (CNL1), Classic Late Infantile Batten Disease (CNL2), Juvenile Batten Disease (CNL3), Batten form CNL4, Batten form CNL5, Batten form CNL6, Batten form CNL7, Batten form CNL8, cystinosis, Farber, fucosidosis, Galactosidosialidosis, Gaucher disease type 1, Gaucher disease type 2, Gaucher disease type 3, GM1 gangliosidosis, Hunter disease, Krabbe disease, α-mannosidosis disease, β-mannosidosis disease, Maroteaux-Lamy, metachromatic leukodystrophy disease, Morquio A, Morquio B, mucolipidosisII/III disease, Niemann-Pick A disease, Niemann-Pick B disease, Niemann-Pick C disease, Pompe disease, Sandhoff disease, SanfiUipo A disease, SanfiUipo B disease, SanfiUipo C disease, SanfiUipo D disease, Schindler disease, Schindler-Kanzaki, sialidosis, Sly disease, Tay-Sachs disease, and Wolman disease. In some embodiments, the disorder of the CNS is Huntington's disease, Parkinson's disease, Alzheimer's disease or frontotemporal dementia.

The invention may also provide the use of an AAV vector and an antimalarial agent, or a pharmaceutical composition according to the invention, for the preparation of a medicament for treating or preventing a disease or disorder in a subject, in particular for the treatment of a disease or disorder of the eye.

The invention may also provide the use of an AAV vector and an antimalarial agent, or a pharmaceutical composition according to the invention, for the preparation of a medicament for the treating or preventing a disease or disorder of the CNS.

In a further aspect, the invention provides an antimalarial agent for use in a method of gene therapy in combination with an AAV vector.

In a yet further aspect, the invention may provide the use of an antimalarial agent to improve the efficiency of transduction of a viral vector into a cell. The cell may be a cell of an organ or tissue. The cells may be retinal cells. The antimalarial agent may be any agent described herein. The viral vector may be any vector described herein. In an embodiment the antimalarial agent is hydroxychloroquine. In an embodiment the viral vector is an AAV vector. In a preferred embodiment the antimalarial agent is hydroxychloroquine and the viral vector is an AAV vector.

According to another aspect, the invention provides a kit for use in increasing the efficiency of transduction of a viral vector, wherein the kit comprises a viral vector to be transduced and an antimalarial agent. The viral vector and antimalarial agent may be provided in the same composition.

Alternatively, in the kit the viral vector may be provided in a first container and the antimalarial agent may be provided in a second container. The kit may comprise instructions to mix the contents of the first and second containers prior to administration. Alternatively, the kit may comprise instructions regarding when to administer the antimalarial agent, and when to administer the viral vector.

The kit may include a syringe for use in injecting the viral vector and/or the antimalarial agent. The kit may be stored either refrigerated or at room temperature.

The skilled man will appreciate that preferred features of any one embodiment and/or aspect and/or claim of the invention may be applied to all other embodiments and/or aspects and/or claims of the invention.

The present invention will be further described in more detail, by way of example only, with reference to the following figures in which:

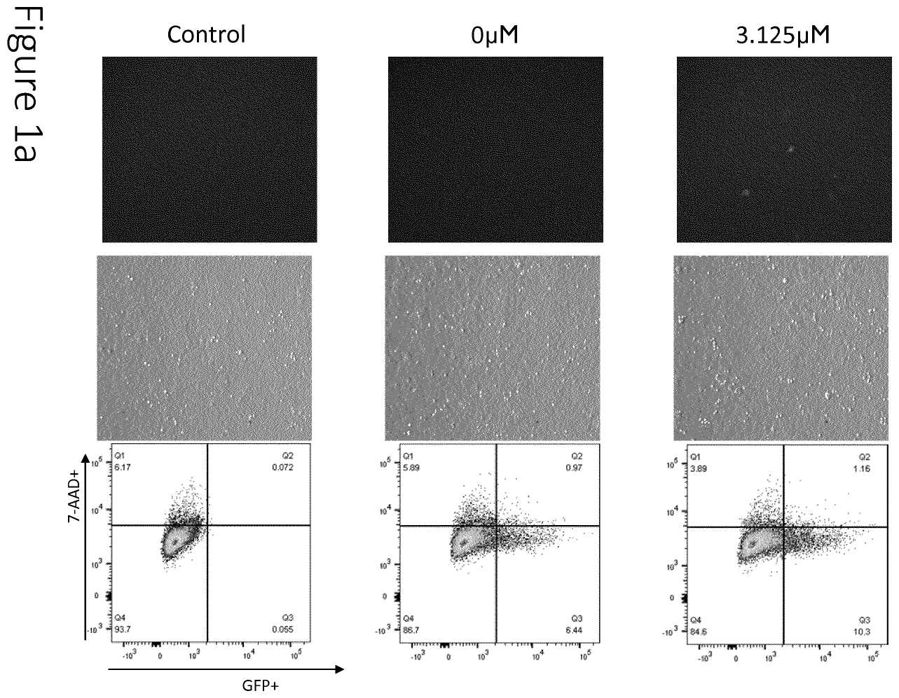

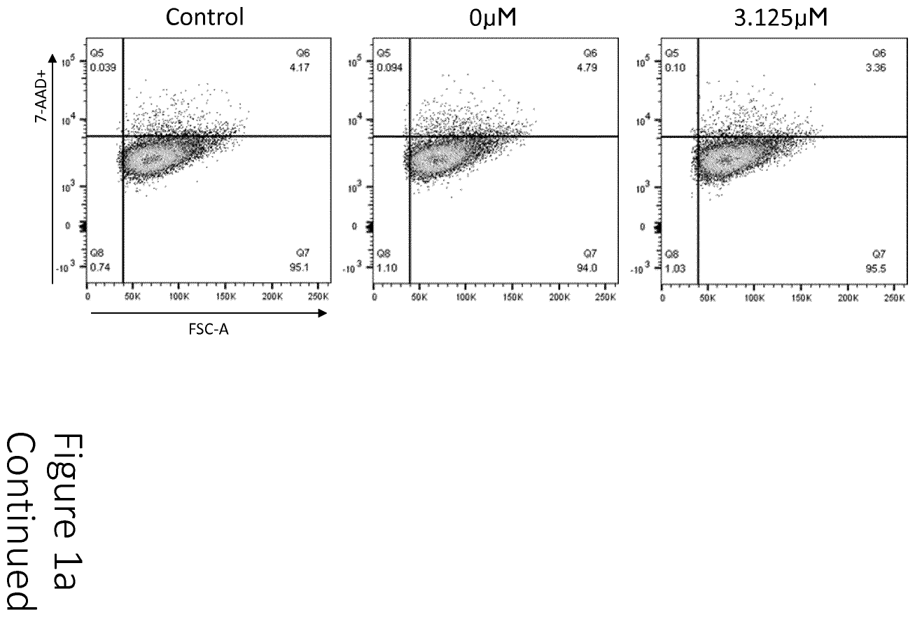

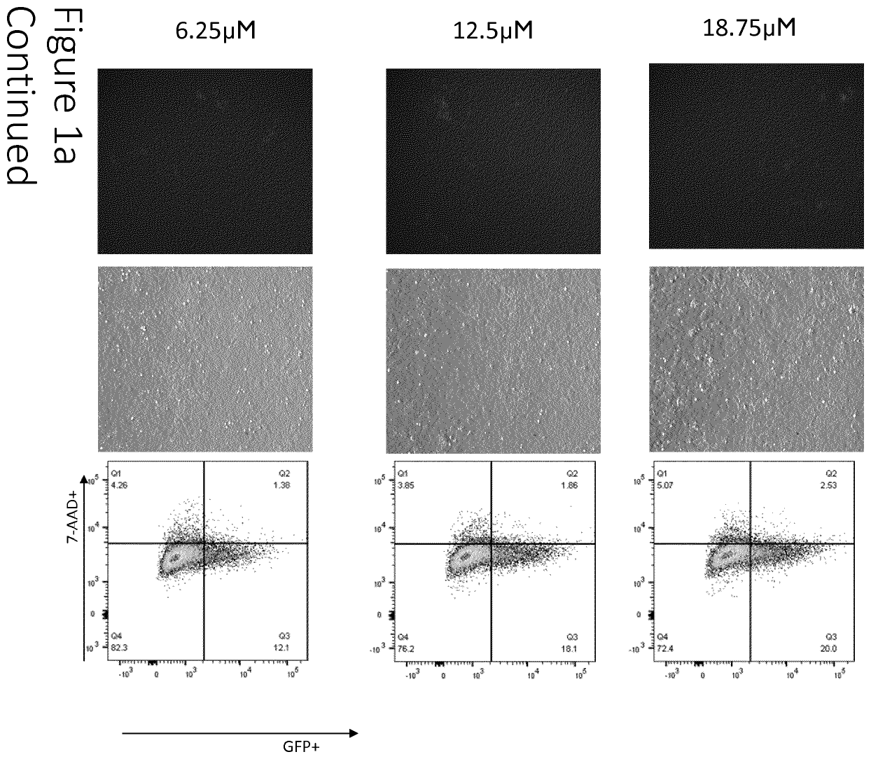

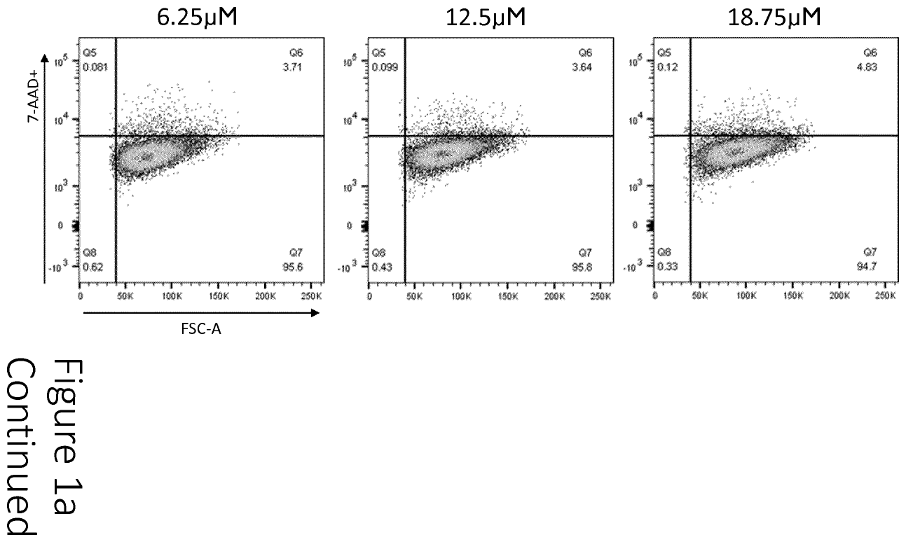

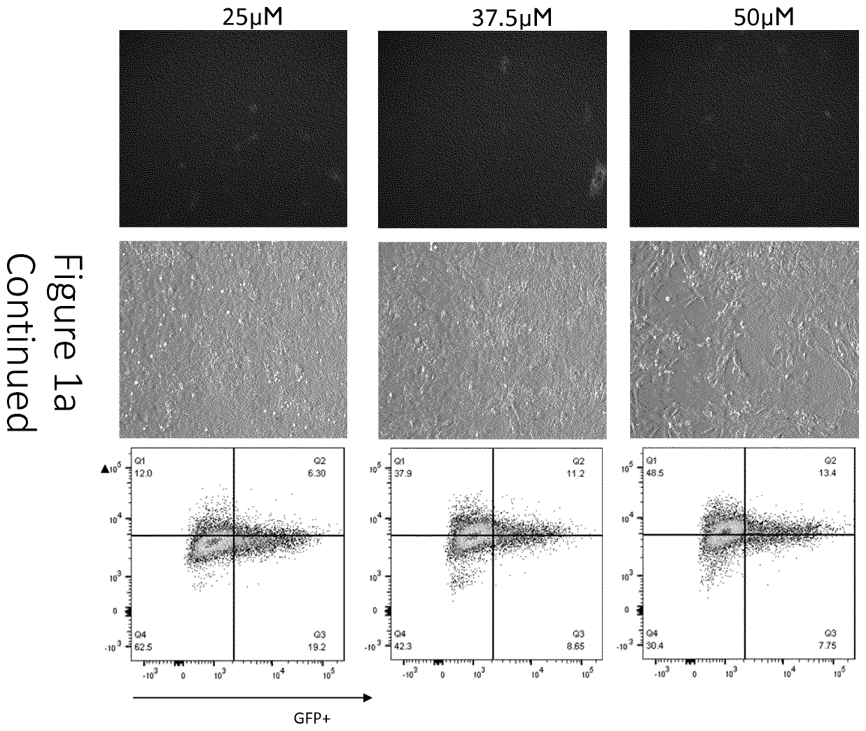

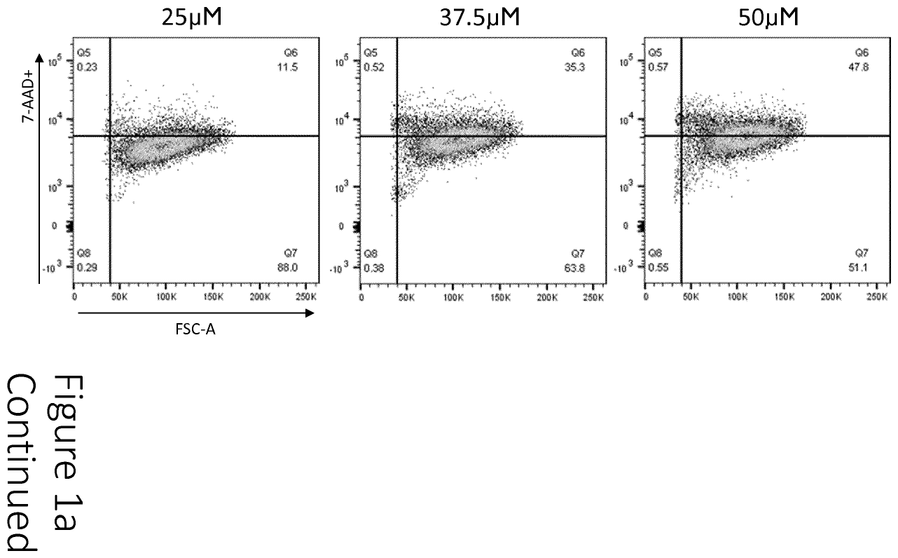

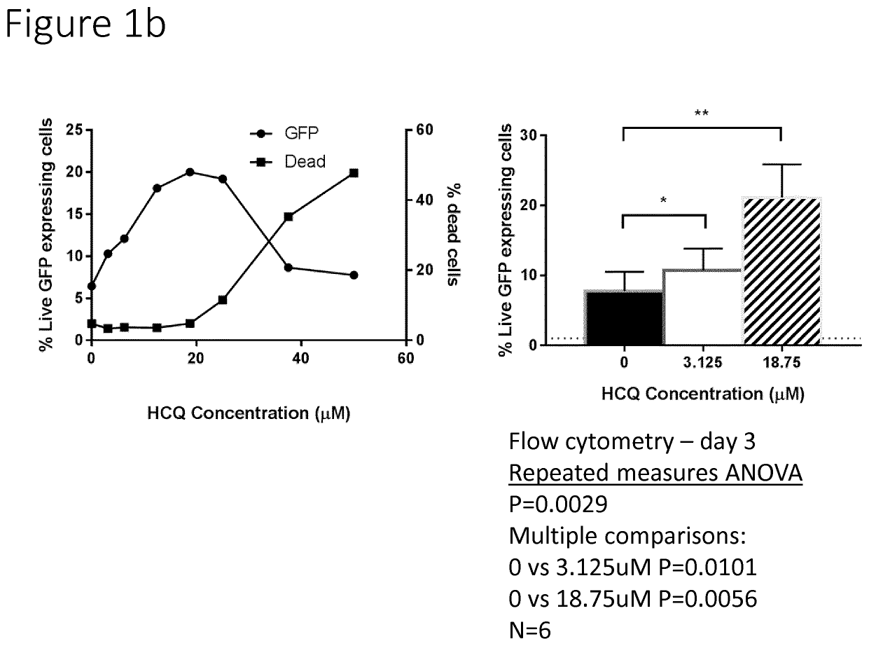

FIGS. 1a and 1b illustrate the effect of hydroxychloroquine (HCQ) on AAV transduction in mouse embryonic fibroblasts (MEFs) and demonstrate the effect to be dose dependent.

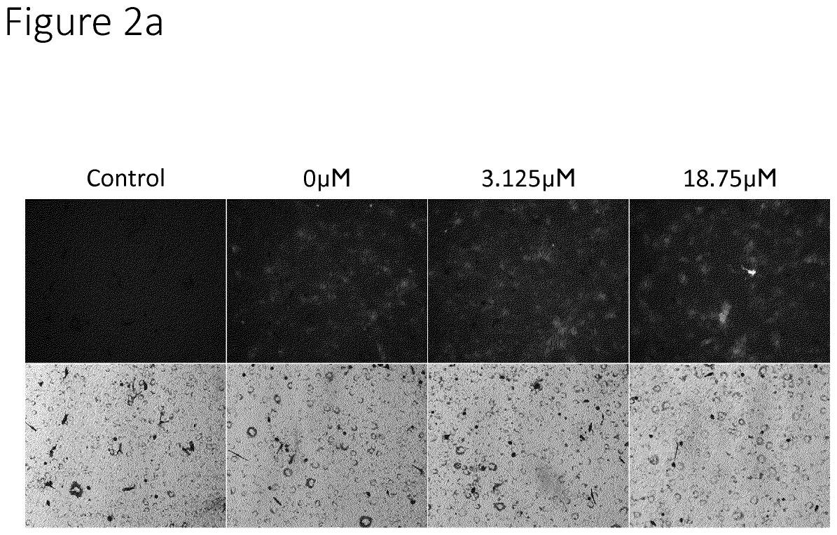

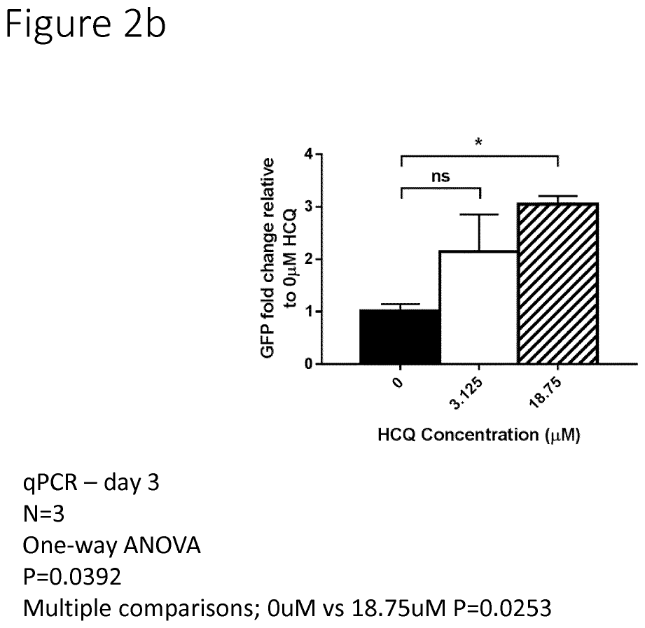

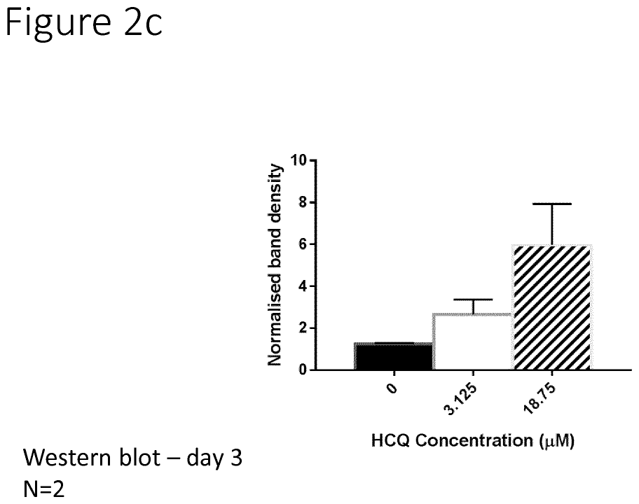

FIGS. 2a, 2b and 2c illustrate the effect of hydroxychloroquine (HCQ) on AAV transduction in non-human primate (NHP) retinal pigment epithelium (RPE). The NHP RPE was obtained from Rhesus macaque (M. mullata) eyes obtained from the MRC Centre for Macaques (Porton Down, UK)

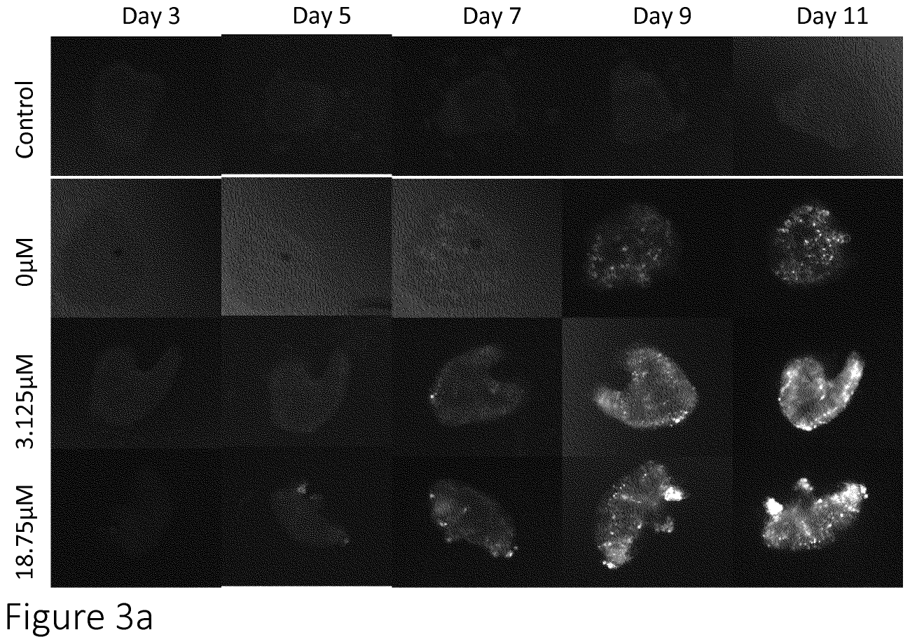

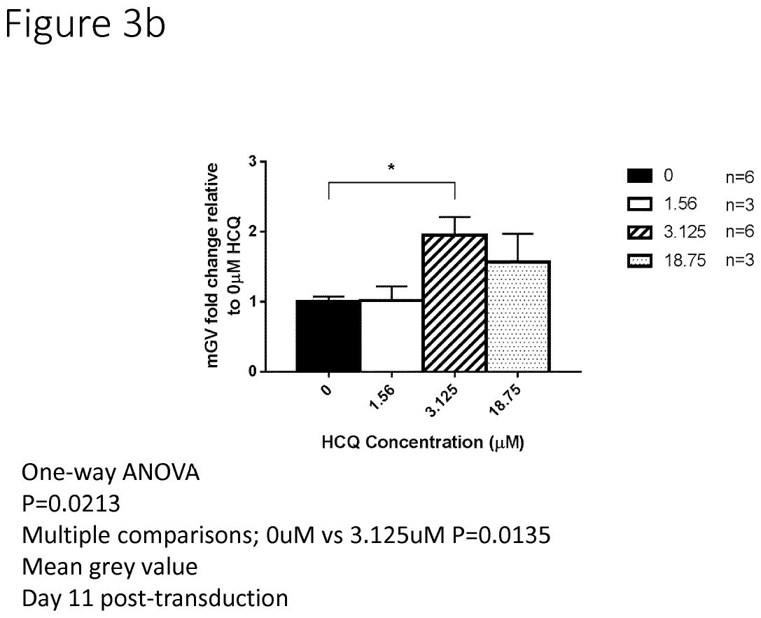

FIGS. 3a and 3b illustrate the effect to hydroxychloroquine (HCQ) on AAV transduction in human retinal explants.

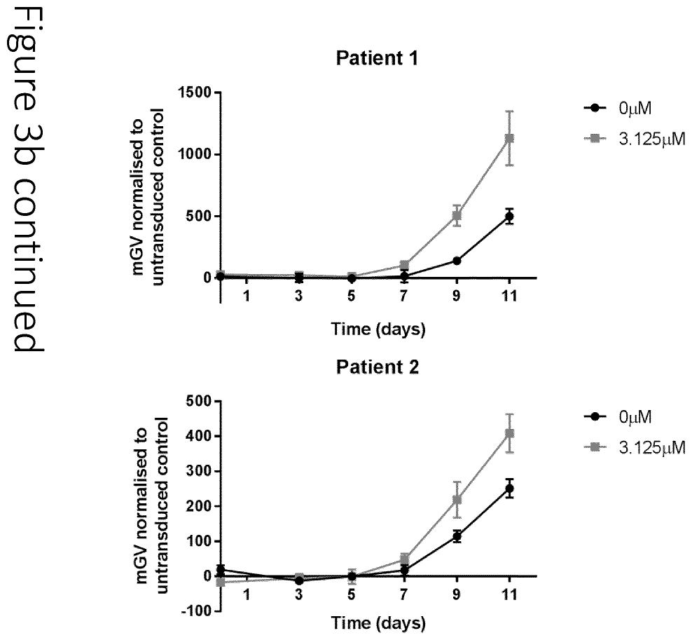

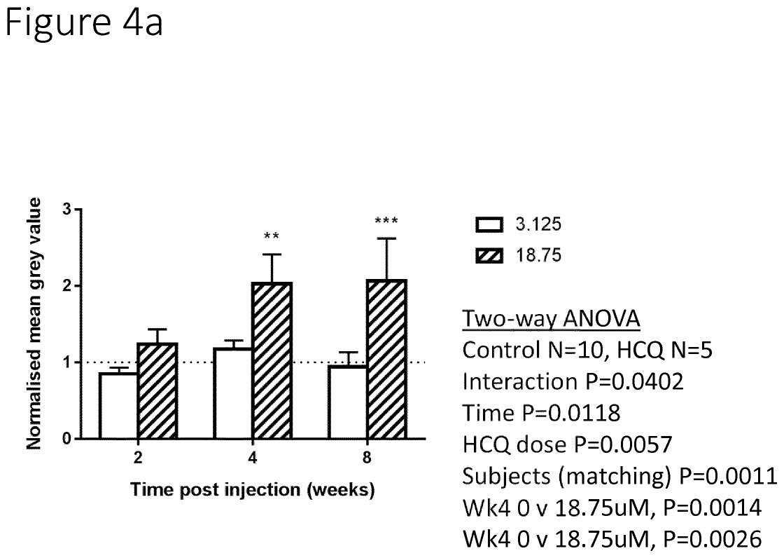

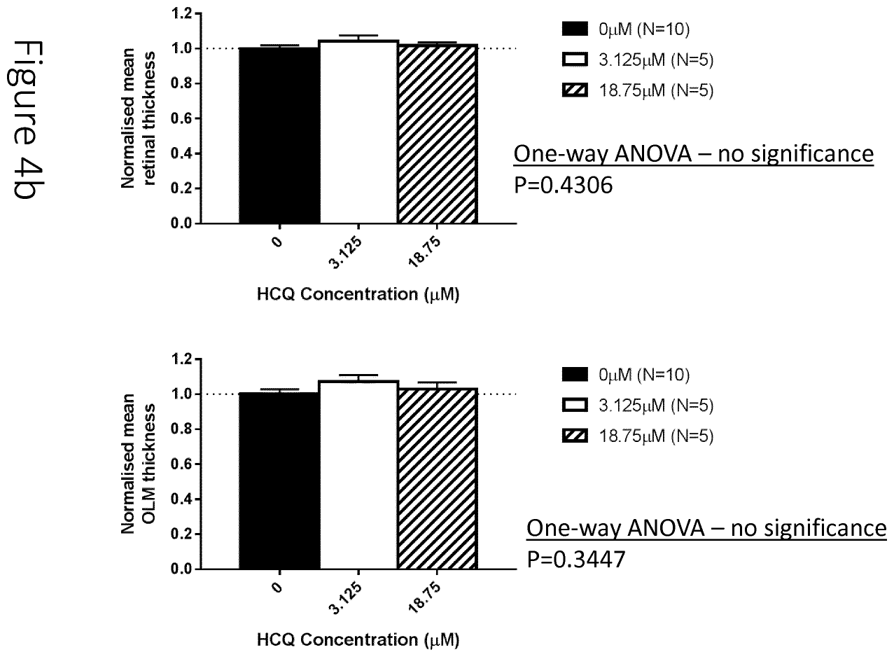

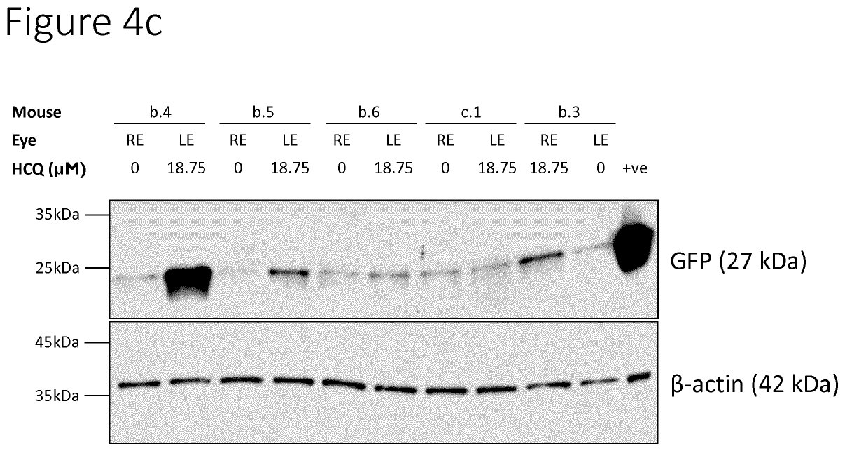

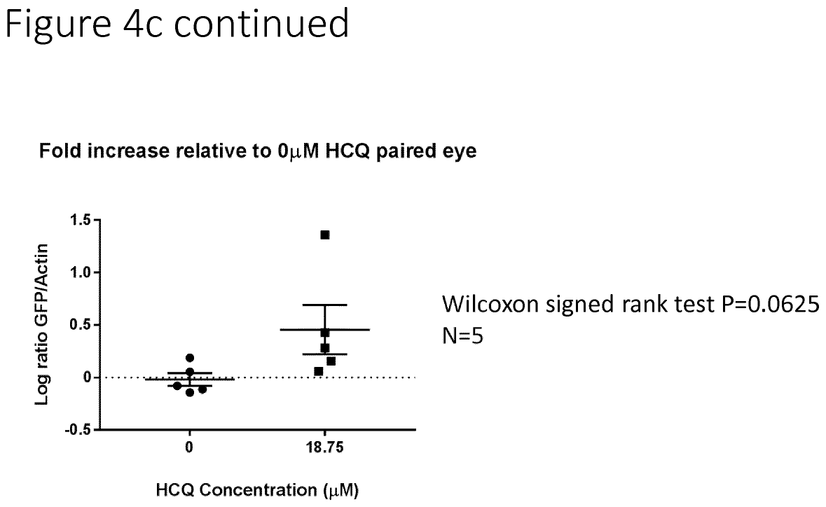

FIGS. 4a, 4b and 4c illustrate that hydroxychloroquine (HCQ) improves the efficacy of AAV transduction in vivo following co-administration of HCQ and an AAV vector encoding green fluorescent protein (GFP) into the subretinal space in mice.

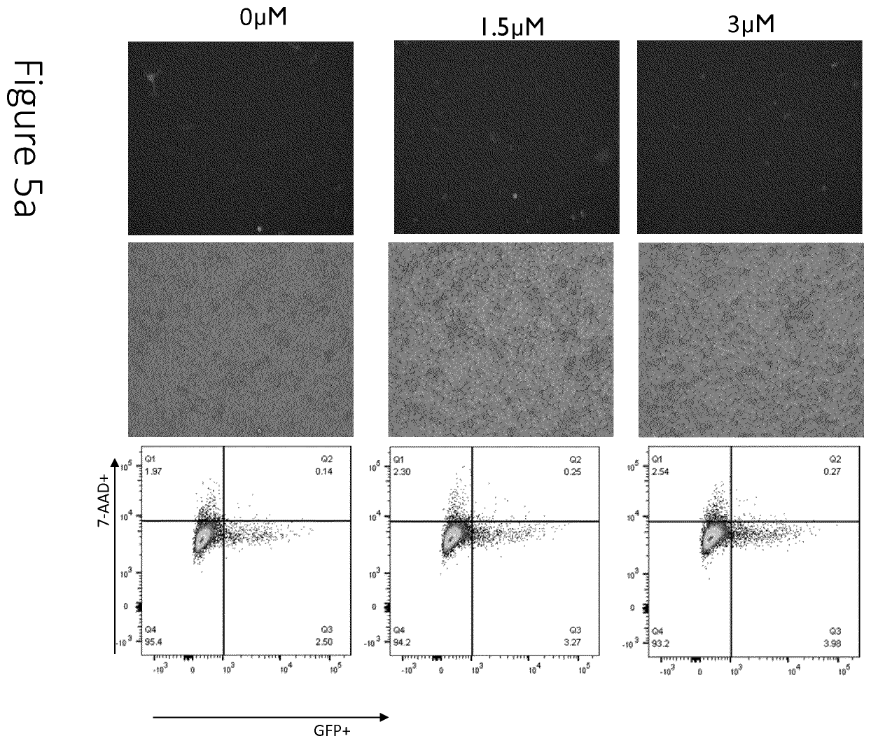

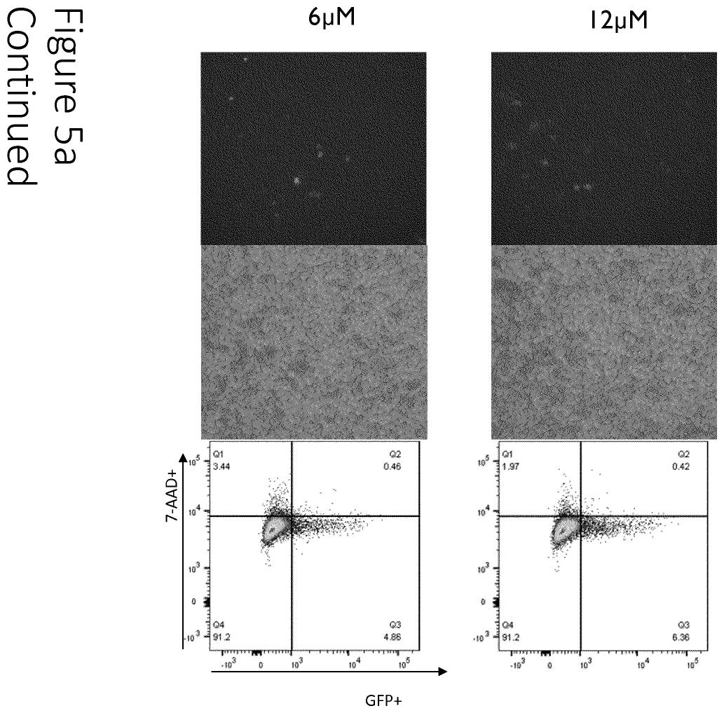

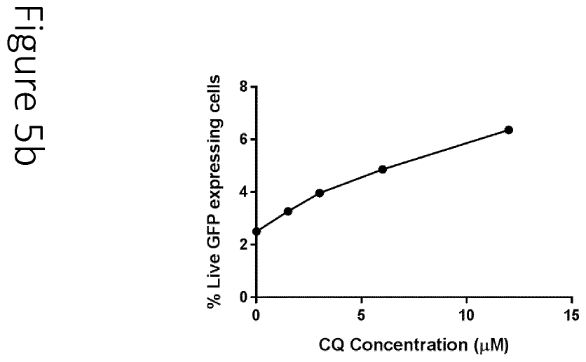

FIGS. 5a and 5b illustrate the effect of chloroquine (CQ) on AAV transduction and demonstrate the effect to be dose dependent.

### EXAMPLE 1

Demonstrates that hydroxychloroquine (HCQ) improves the efficacy of AAV transduction in a dose dependent manner.

Methods:

Cell culture. Wildtype (WT) mouse embryonic fibroblasts (MEFs) were seeded into 6-well plates. 24 hr after seeding the cells were incubated in culture media with HCQ concentrations of 3.125-50 uM for 1 hr prior to transduction with a rAAV2.CAG.GFP.WPRE.pA vector at a MOI of 1000. The cells were cultured in HCQ containing media until harvesting. Cells were imaged 3 days post-transduction (FIG. 1A).

Flow cytometry analysis. Cells were processed 3 days post-transduction and stained with the cell viability dye 7-AAD for 5 minutes prior to flow cytometry analysis. Fluorescent light was measured by a BD LSRFortessa flow cytometer using a blue laser with the bandpass filters 695 nm/40 mW and 530 nm/30 mW to detect the fluorochromes of 7-AAD and GFP, respectively (FIG. 1a).

Results & Discussion:
(a) The results presented in FIGS. 1a and 1b demonstrate that increasing concentrations of HCQ increase the number of GFP expressing cells up to 18.75 uM. At concentrations of 25 uM and above HCQ induces cell death which in turn decreases the proportion of live GFP expressing cells.
(b) The results also demonstrate that HCQ increases the number of GFP expressing cells in a dose dependent manner in WT MEFs.

The results presented demonstrate that the antimalarial agent HCQ improves the efficacy of AAV transduction.

### EXAMPLE 2

Demonstrates that HCQ improves the efficacy of AAV transduction in non-human primate (NHP) retinal pigment epithelium (RPE) cells

Methods:

Harvesting non-human primate (NHP) retinal pigment epithelium (RPE) cells. After enucleation, the cornea and lens were removed under direct visualization with a surgical microscope. Radial incisions were made towards the posterior pole to flatten the eyecup. The retina was removed by blunt dissection and the remaining eyecup was placed in media and stored on ice until the RPE cells were removed. RPE cells were detached using the TrypLE cell dissociation reagent and were cultured at 37° C.

Cell culture. Primary NHP RPE cells were seeded into a 12-well plate. 24 hr after seeding, the cells were incubated in media with 3.125 uM or 18.75 uM of HCQ for 1 hr prior to transduction with 2×10⁹ genome copies per well. Cells were imaged 3 days post-transduction.

RNA & protein extraction and cDNA synthesis. The primary NHP RPE were harvested for RNA extraction 3-days post-AAV transduction with HCQ. Total RNA was extracted using the RNeasy Mini Kit (Qiagen) according to the manufacturer's instructions. Copy DNA was synthesised using 850-1500 ng of RNA using the Superscript III Kit (Invitrogen) with an oligo dT primer.

qPCR analysis. qPCR was carried out using TaqMan probes against GFP and β-actin in a CFX Connect thermal cycler (BioRad). GFP expression was normalized to β-actin and represented as a fold change to transduced cells incubated without HCQ.

Protein extraction. The flow through from RNA extraction above was combined with 4 volumes of acetone and incubated on ice for 30 min. Samples were centrifuged at 17,000×g for 30 min, the supernatant was discarded and the pellet washed in 100 μL of ice-cold ethanol. Pellets were resuspended in 1% SDS.

Western blot. Total protein content in cell lysates was determined using the bicinchoninic acid method. GFP and actin proteins were probed using monoclonal antibodies, and detected using ECL and Odyssey Imaging System (LI-COR). Data analysis was performed using ImageStudio Lite software (LI-COR). GFP band density was normalised to β-actin.

Results & Discussion:

GFP mRNA expression was increased by 3 fold (N=3, p=0.0253) and GFP protein expression increased by 19 fold at 3 days post-transduction when primary NHP RPE cells were treated with 18.75 μM of HCQ compared to transduced cells incubated without HCQ (FIGS. 2a and 2b). These results demonstrate that 18.75 μM of HCQ improved AAV transduction in the primary NHP RPE cells.

Protein was extracted from the RNA extraction flow through, however this yielded low concentrations so it was only possible to run a western blot on N=2, which showed that 18.75 μM of HCQ improved GFP protein expression by approximately 4-fold in the NHP RPE cells (FIG. 2c).

### EXAMPLE 3

Demonstrates that HCQ improves the efficacy of AAV transduction in human retinal explants.

Methods:

Harvesting tissue. Human retina fragments was extracted during routine retinal surgery in which a retinectomy was required (ethics approved). Fragments of retina were removed via a 23G sclerostomy with vitrectomy cutter (at minimal cut rate) and manual aspiration.

Culturing explants. Within 1 hr of tissue collection, retinal fragments were transferred using a 3 mL Pasteur pipette into individual organotypic culture inserts (BD Falcon), which were in turn placed within a 24-well plate. 400 uL of media was placed beneath the insert and 100 uL of media within the insert. 24 hr after culturing the retinal explants, the media was replaced with media containing 3.125 uM or 18.75 uM of HCQ. The explants were incubated in the HCQ medium for 1 hour prior to transduction with 1×10⁹ genome copies of rAAV2.CAG.GFP.WPRE.pA vector. The explants were imaged every 2 days for 11 days and the mean grey value (representative of GFP fluorescence) of the explants was measured using ImageJ software (FIG. 3a).

Results & Discussion:

The AAV transduced explants demonstrated an up to 2-fold increase in mean grey value when treated with 3.125 uM HCQ compared to with no HCQ (n=6, p=0.0213) (FIG. 3b). This demonstrates that 3.125 μM of HCQ improved AAV transduction.

### EXAMPLE 4

Illustrate that hydroxychloroquine (HCQ) improves the efficacy of AAV transduction in vivo following co-administration of HCQ and an AAV vector encoding green fluorescent protein (GFP) into the subretinal space in mice.

Methods:

Subretinal injections. 7 week old female C57BL/6 mice were subretinally injected with 1.2 μL vector suspension (1×10⁸ genome copies). The vector was prepared with a 3.125 uM or 18.75 uM final concentration of HCQ with control preparations without any HCQ. Animals were injected with an AAV+HCQ preparation in one eye and AAV only in the paired eye.

Confocal scanning laser ophthalmoscope (cSLO) analysis. Standardized mouse autofluorescence (AF) imaging using a cSLO (Spectralis HRA, Heidelberg Engineering) was performed in all animals according to previously published protocol. All images were recorded using the 55° lens of the Spectralis HRA at 2, 4 and 8 weeks post-injection. Images were recorded using a standardized signal detector sensitivity. For quantitative analysis of fundus AF, the mean grey level was measured within a ring-shaped area located at radii between 350 and 860 pixels from the optic disc centre using ImageJ software (NIH).

OCT analysis. Animals were subject to wide-field spectral-domain optical coherence tomography (OCT, Spectralis HRA, Heidelberg Engineering) at 2, 4 and 8 weeks post-injection. Mice were scanned using a 55° lens and 8 radial sections were taken with a real-time average process of 25 frames. The total retinal thickness and the outer limiting membrane (OLM) thickness were manually measured on alternate radial sections using a calliper.

Protein extraction. Mouse retinas were thawed on ice and lysed in 1× RIPA buffer+protease inhibitors. The retinas were homogenised using a hand-held homogeniser and incubated on ice for 30 mins. The samples were centrifuged at 17,000×g at 4° C. for 20 mins. The supernatant was transferred to a new tube and kept on ice.

Western blot (WB). Total protein content in cell lysates was determined using the bicinchoninic acid method. GFP and actin proteins were probed using monoclonal antibodies, and detected using ECL and Odyssey Imaging System (LI-COR). Data analysis was performed using ImageStudio Lite software (LI-COR). GFP band density was normalised to actin.

Results & Discussion:

Quantification of the mean grey value of the fundus AF images demonstrated a statistically significant 2-fold increase in mean grey value (a surrogate measure of GFP expression) in the eyes subretinally injected with a suspension of AAV and 18.75 uM HCQ at 4 and 8 weeks post-injection compared to control eyes injected with AAV only (FIG. 4a).

There was no significant difference in the mean retinal thickness or retinal morphology between any of the groups with and without HCQ in the subretinal injection suspension (FIG. 4b). This suggests that subretinal HCQ did not have any detectable toxic effect on the retina of the animals.

FIG. 4c illustrates that there was an increase in the GFP signal detected by WB compared to the 0 μM HCQ paired eye, demonstrating that 18.75 μM HCQ increases GFP protein levels.

### EXAMPLE 5

Demonstrates that chloroquine (CQ), like HCQ in Example 1, improves the efficacy of AAV transduction in a dose dependent manner.

Methods:

Cell culture. Wildtype (WT) mouse embryonic fibroblasts (MEFs) were seeded into 6-well plates. 24 hr after seeding, the cells were incubated in media with CQ concentrations of 1.5 to 12 uM for 1 hr prior to transduction with a rAAV2.CAG.GFP.WPRE.pA vector at a MOI of 1000. The cells were cultured in CQ containing media until harvesting. Cells were imaged 3 days post-transduction (FIG. 5a).

Flow cytometry analysis. Cells were processed 3 days post-transduction and stained with the cell viability dye 7-AAD for 5 min prior to flow cytometry analysis. Fluorescent light was measured by a BD LSRFortessa using a blue laser with bandpass filters 695 nm/40 mW and 530 nm/30 mW to detect the fluorochromes of 7-AAD and GFP, respectively (FIG. 5a).

Results & Discussion:

The results presented in FIGS. 5a and 5b demonstrate that increasing concentrations of CQ increase the number of GFP expressing cells up to 12 uM. The results also demonstrate that CQ increases the number of GFP expressing cells in a dose dependent manner.

The results presented demonstrate that the antimalarial agent, CQ, improves the efficacy of AAV transduction.

1. A method of improving the efficiency of transduction of viral vectors into cells, wherein the method comprises administering to a cell an antimalarial agent and a viral vector.

2. The method of claim 1 wherein the antimalarial agent and viral vector are administered simultaneously, sequentially or separately.

3. The method of claim 1 or claim 2 wherein the antimalarial agent is selected from the group comprising or consisting of 4-aminoquinolines (such as hydroxychloroquine, chloroquine and amodiaquine), 8-aminoquinolines (such as primaquine, pamaquine and tafenoquine), mefloquine, quinine, mepacrine, atovaquone, doxycycline, and a salt or derivative thereof.

4. The method of any preceding claim wherein the antimalarial agent is a quinoline compound.

5. The method of claim 4 wherein the antimalarial agent is selected from the group comprising or consisting of hydroxychloroquine, chloroquine, mefloquine, amodiaquine, quinine, pamaquine, primaquine, mepacrine, and a salt, or a derivative thereof.

6. The method of claim 5 wherein the antimalarial agent is hydroxychloroquine.

7. The method of any preceding claim wherein the viral vector is selected from the group comprising or consisting of an adeno-associated virus (AAV), an adenovirus, a retrovirus, a lentivirus, a vaccinia/poxvirus, or a herpesvirus.

8. The method of claim 7 wherein the viral vector is an adeno-associated viral (AAV) vector.

9. The method of any preceding claim wherein the antimalarial agent is hydroxychloroquine and the viral vector is an adeno-associated viral (AAV) vector.

10. The method of any preceding invention wherein the method is carried out in vitro or in vivo.

11. The method of any preceding claim wherein the antimalarial agent and/or viral vector are administered systemically or locally; and/or
wherein the antimalarial agent and the viral vector are administered to the same cells, tissue or organ; and/or
wherein the antimalarial agent and the viral vector are co-administered; and/or
wherein the antimalarial agent and/or viral vector are administered at the intended site of transduction.

12. The method of any preceding claim wherein the viral vectors carry a cargo which is expressed upon transduction.

13. The method of any preceding claim wherein more than one viral vector is administered.

14. The method of claim 12 wherein the cargo comprises a transgene or part thereof intended to treat a disease or disorder; or
wherein the cargo comprises at least one component needed to facilitate CRISPR gene editing; or
wherein the cargo comprises an inhibitory RNA or a Mirtron; or
wherein the cargo comprises one or more components needed to deliver an optogenetic therapy or system to a cell, tissue or organ.

15. A method of any of claims 1 to 9 wherein the method is used to increase the yield of recombinant AAV particles during a production run of the viral vector.

16. An antimalarial for use in increasing the transduction efficiency of a viral vector into a cell.

17. The antimalarial for the use of claim 16 wherein the antimalarial agent is hydroxychloroquine and the viral vector is an adeno-associated viral (AAV) vector.

18. A composition comprising a viral vector and an antimalarial agent.

19. A pharmaceutical composition comprising a viral vector, an antimalarial agent, and a pharmaceutically acceptable carrier, diluent or excipient.

20. A pharmaceutical composition according to claim 34 wherein the composition comprises an AAV vector, and one or more of hydroxychloroquine, chloroquine, and mefloquine.

21. A pharmaceutical composition according to claim 19 or 20 wherein the composition is intended for ocular administration.

22. The composition or pharmaceutical composition according to any of claims 18 to 21, the use of claim 16 or 17, or the method of any of claims 1 to 15, for use in the treatment of a disease or disorder.

23. The composition or pharmaceutical composition, or use, or method, according to claim 22 wherein the disease or disorder is a disease or disorder of the eye; or
where the composition, use, or method, is to enhance the gene therapy treatment of an ocular disorder or disease, and optionally wherein the viral vector and/or antimalarial agent are administered directly to the eye, for example by subretinal, suprachoroidal, intravitreal, peri-ocular or anterior chamber injection.

24. The composition or pharmaceutical composition, or use, or method, according to claim 22 wherein the disease or disorder is a CNS condition, and optionally wherein the AAV vector and/or the antimalarial agent are administered by direct spinal cord injection and/or intracerebral administration.

25. The composition or pharmaceutical composition, or use, or method, according to claim 22 wherein the proportion of cells transduced by the viral vector in a target cell population is increased by at least 1% in the presence of the antimalarial agent compared to using the viral vector alone; or
wherein the proportion of cells transduced by the viral vector in a target cell population is increased by at least 1 fold in the presence of the antimalarial agent compared to using the viral vector alone.

26. The composition or pharmaceutical composition according to any of claims 18 to 21, the use of claim 16 or 17, or the method of any of claims 1 to 15 wherein the antimalarial agent is used at a concentration of between about 1 μM and about 30 μM.

27. A method of treating a disease or disorder in a subject, wherein the method comprises administering to the subject a pharmaceutical composition according to claim 19 or 20, optionally the disease or disorder is a disease or disorder of the eye or is a CNS condition.

28. A kit for use in increasing the efficiency of transduction of a viral vector, wherein the kit comprises a viral vector to be transduced and an antimalarial agent.

29. The kit of claim 28 wherein the viral vector and antimalarial agent are provided in the same composition.

30. The kit of claim 41 wherein the viral vector is provided in a first container and the antimalarial agent is provided in a second container, and optionally further comprising instructions to mix the contents of the first and second containers prior to administration, and/or
further comprising instructions when to administer the antimalarial agent, and when to administer the viral vector.

31. The kit of any of claims 28 to 30 further comprising one or more syringes for use in injecting the viral vector and/or the antimalarial agent.
